(19) United States
(12) Patent Application Publication
(10) Pub. No.: US 2015/0025517 A1
(43) Pub. Date: Jan. 22, 2015

(54) PROBE AND TREATMENT INSTRUMENT INCLUDING PROBE

(71) Applicant: OLYMPUS MEDICAL SYSTEMS CORP., Tokyo (JP)

(72) Inventor: Yukihiko SAWADA, Yoshikawa-shi (JP)

(21) Appl. No.: 14/330,377

(22) Filed: Jul. 14, 2014

Related U.S. Application Data
(60) Provisional application No. 61/847,775, filed on Jul. 18, 2013.

(51) Int. Cl. A61B 17/32; A61B 18/08

(52) U.S. Cl. CPC A61B 17/320092; A61B 18/08
USPC 606/28; 606/169

(57) ABSTRACT

A probe treating a living tissue in cooperation with a jaw pivoting on a supporting point includes a treatment portion including a proximal end to which ultrasonic vibration generated from an ultrasonic transducer unit is transmitted; a distal end to which the ultrasonic vibration is transmitted from the proximal end; a holding surface holding the living tissue between the holding surface and the jaw; a back surface serving as a rear surface of the holding surface; a side surface extending from the proximal end to the distal end and being different from the holding surface; and a stress release portion provided between the distal end and the proximal end and on the side surface and releasing stress to stop development of a crack when the crack is produced in the holding surface or the back surface by external forces applied during the treatment of the living tissue.

### CROSS-REFERENCE TO RELATED APPLICATIONS

This application claims the benefit of U.S. Provisional Application No. 61/847,775, filed Jul. 18, 2013, the entire contents of which are incorporated herein by reference.

### BACKGROUND OF THE INVENTION

1. Field of the Invention

This invention relates to a probe to which ultrasonic vibration is input, and a treatment instrument including the probe.

2. Description of the Related Art

For example, U.S. Pat. No. 8,048,074 B2 has disclosed a probe capable of transmitting ultrasonic vibration from its proximal end to its distal end to treat a living tissue at the distal end. The probe is attached to a treatment instrument body together with an ultrasonic transducer unit in use.

### BRIEF SUMMARY OF THE INVENTION

In an aspect of the present invention, a probe configured to treat a living tissue in cooperation with a jaw which is configured to pivot on a supporting point includes a treatment portion including a proximal end to which ultrasonic vibration generated from an ultrasonic transducer unit is transmitted; a distal end to which the ultrasonic vibration is transmitted from the proximal end; a holding surface configured to hold the living tissue between the holding surface and the jaw; a back surface serving as a rear surface of the holding surface; a side surface which extends from the proximal end to the distal end and which is different from the holding surface; and a stress release portion which is provided between the distal end and the proximal end and on the side surface and which releases stress to stop development of a crack when the crack is produced in the holding surface or the back surface by external forces applied during the treatment of the living tissue.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic diagram showing an ultrasonic treatment system according to first and second embodiments;

FIG. 2 is a schematic cross sectional view showing a connection between an ultrasonic transducer unit and a probe in the ultrasonic treatment system according to the first and second embodiments;

FIG. 3 is a schematic diagram showing the probe in the ultrasonic treatment system according to the first and second embodiments;

FIG. 4 is a schematic side view showing a treatment portion of the probe in the ultrasonic treatment system according to the first embodiment;

FIG. 5A is a schematic cross sectional view of the treatment portion of the probe in the ultrasonic treatment system according to the first embodiment taken along the line 5A-5A in FIG. 4;

FIG. 5B is a schematic cross sectional view of the treatment portion of the probe in the ultrasonic treatment system according to the first embodiment taken along the line 5B-5B in FIG. 4;

FIG. 5C is a schematic cross sectional view of the treatment portion of the probe in the ultrasonic treatment system according to the first embodiment taken along the line 5C-5C in FIG. 4;

FIG. 6A is a schematic side view showing the treatment portion of the probe in the ultrasonic treatment system according to the first embodiment, and is a schematic diagram showing how a holding surface to hold a living tissue has cracked and the growth of the crack is blocked in a stress release portion;

FIG. 6B is a schematic side view showing the treatment portion of the probe in the ultrasonic treatment system according to the first embodiment, and is a schematic diagram showing how a back surface opposite to the holding surface to hold the living tissue has cracked and the growth of the crack is blocked in the stress release portion;

FIG. 7A is a schematic cross sectional view showing how the side surfaces of the treatment portion of the probe are pierced obliquely to a direction that intersects at right angles with a rotation surface of a jaw;

FIG. 7B is a schematic cross sectional view showing how the side surfaces of the treatment portion of the probe are pierced obliquely to the direction that intersects at right angles with the rotation surface of the jaw and is then to be pierced at a different oblique angle;

FIG. 7C is a schematic cross sectional view showing how the side surfaces of the treatment portion of the probe are pierced in two directions obliquely to the direction that intersects at right angles with the rotation surface of the jaw;

FIG. 8 is a schematic side view showing the treatment portion of the probe in the ultrasonic treatment system according to a first modification of the first embodiment;

FIG. 9 is a schematic side view showing the treatment portion of the probe in the ultrasonic treatment system according to a second modification of the first embodiment;

FIG. 10 is a schematic side view showing the treatment portion of the probe in the ultrasonic treatment system according to a third modification of the first embodiment;

FIG. 11A is a schematic side view showing the treatment portion of the probe in an ultrasonic treatment system according to the second embodiment;

FIG. 11B is a schematic cross sectional view of the treatment portion of the probe in the ultrasonic treatment system according to the second embodiment taken along the line 11B-11B in FIG. 11A;

FIG. 12A is a schematic side view showing the treatment portion of the probe in the ultrasonic treatment system according to a first modification of the second embodiment;

FIG. 12B is a schematic cross sectional view of the treatment portion of the probe in the ultrasonic treatment system according to the first modification of the second embodiment taken along the line 12B-12B in FIG. 12A; and

FIG. 12C is a schematic bottom view showing the treatment portion of the probe in the ultrasonic treatment system according to the first modification of the second embodiment.

### DETAILED DESCRIPTION OF THE INVENTION

Hereinafter, embodiments of this invention will be described with reference to the drawings.

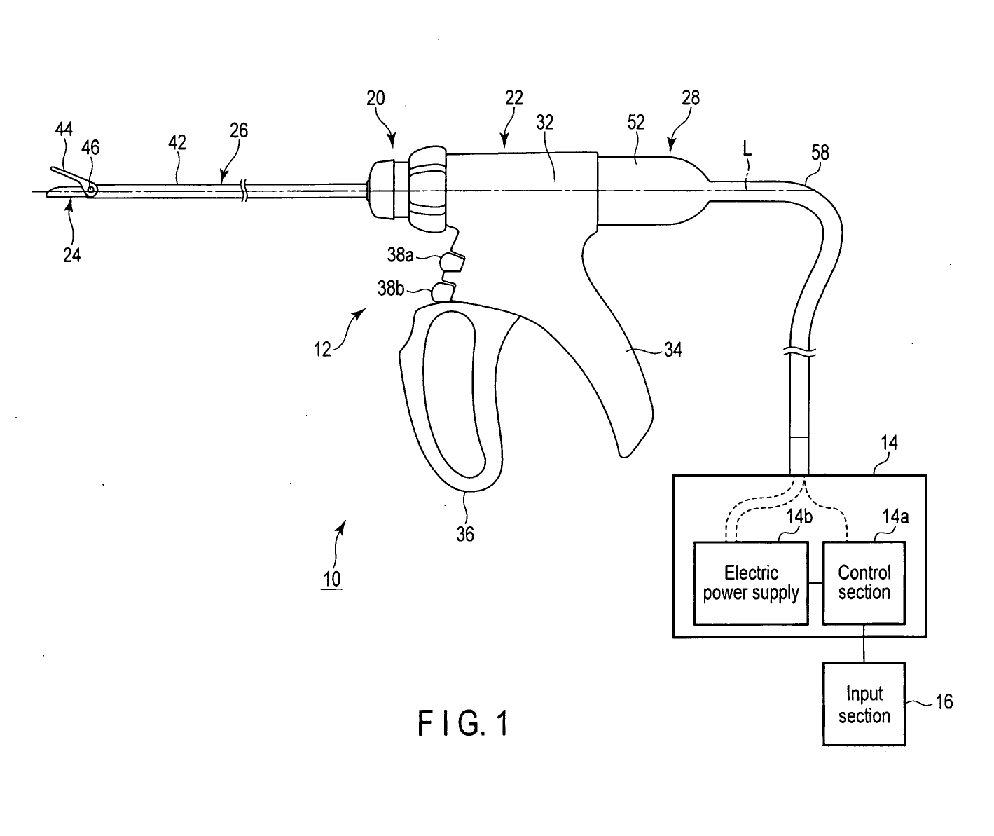

The first embodiment is described with reference to FIG. 1 to FIG. 6B.

As shown in FIG. 1, an ultrasonic treatment system 10 according to this embodiment includes an ultrasonic treatment instrument 12, a power supply unit 14, and an input section 16. The ultrasonic treatment device 12 includes a handle unit 22, a probe 24, a sheath unit 26, and an ultrasonic transducer unit 28. The handle unit 22 and the sheath unit 26 constitute a treatment instrument body 20.

The power supply unit 14 includes a control section 14a, and an electric power supply 14b which supplies electric power to a later-described ultrasonic transducer 54 of the ultrasonic transducer unit 28. The input section 16 is connected to the control section 14a of the power supply unit 14, and instructs whether to supply the electric power to the ultrasonic transducer 54 from the electric power supply 14b. The input section 16 is comprised of, for example, a foot switch.

The handle unit 22 includes a handle body 32 having a fixed handle 34, and a movable handle 36 capable of moving close to or away from the handle body 32. The movable handle 36 is biased by a spring (not shown) to move away from the fixed handle 34 of the handle body 32. The handle body 32 is provided with a coagulation switch 38a and a cut switch 38b. The coagulation switch 38a and the cut switch 38b are connected to the control section 14a. The amount of electric power supplied to the later-described ultrasonic transducer 54 from the electric power supply 14b, for example, is properly controlled by the selection of the switches 38a and 38b.

The sheath unit 26 includes a sheath (tubular body) 42 which covers the outer circumferential surface of the probe 24 and which is attachable to and detachable from the handle body 32 of the handle unit 22, a jaw 44 provided at the distal end of the sheath 42, and a supporting point 46 which rotatably supports the jaw 44 at the distal end of the sheath 42. The jaw 44 is capable of pivoting on the supporting point 46. The jaw 44 is located to face a later-described treatment portion 74 of the probe 24. The jaw 44 is rotatable relative to the supporting point 46 disposed at the distal end of the sheath 42 by the use of a known means in response to the operation of the movable handle 36. More specifically, when the movable handle 36 is located away from the fixed handle 34, the jaw 44 is located away from the treatment portion 74, that is to say, is in an open state. If the movable handle 36 is moved closer to the fixed handle 34, the later-described jaw 44 of the sheath unit 26 is moved closer to the treatment portion 74 of the probe 24, that is to say, into a closed state.

It is preferable that the position of the jaw 44 facing the later-described treatment portion 74 of the probe 24, and the inner circumferential surface and outer circumferential surface of the sheath 42 are covered with a heat-resistant and electrically insulating material. A fluorocarbon resin such as PTFE is suitably used as the heat-resistant and electrically insulating material.

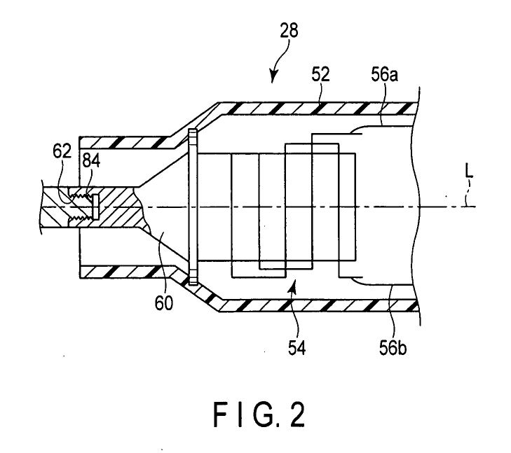

As shown in FIG. 2, the ultrasonic transducer unit 28 includes a transducer case 52 which is made of, for example, an insulating resin material and which serves as an outer shell, the ultrasonic transducer 54 which is a vibration generator disposed inside the transducer case 52, electric wiring lines 56a and 56b connected at one end to the ultrasonic transducer 54, and a cable 58 which extends from the proximal end of the transducer case 52 and which removably connects the electric wiring lines 56a and 56b to the electric power supply 14b of the power supply unit 14. An unshown connector of the cable 58 is removably connected to the power supply unit 14. Various signal lines of, for example, switches 38a and 38b are provided inside the cable 58 in addition to the electric wiring lines 56a and 56b. The signal lines of the switches 38a and 38b and a signal line of the ultrasonic transducer unit 28 are electrically connected to each other when the ultrasonic transducer unit 28 is coupled to the handle unit 22.

For example, the ultrasonic transducer 54 of a BLT type is used. The electric power supply 14b supplies electric power to the ultrasonic transducer 54 via the electric wiring lines 56a and 56b inside the cable 58. For example, a bolt-clamped Langevin type transducer which resonates at a half wavelength (λ/2) is used as the ultrasonic transducer 54. If electric energy is applied to the ultrasonic transducer 54 from the power supply unit 14 through the cable 58, the electric energy is converted to ultrasonic vibration of mechanical energy by the ultrasonic transducer 54.

A horn 60 which increases the amplitude of the ultrasonic vibration is coupled to the distal-direction side of the ultrasonic transducer 54. The horn 60 is attached to, for example, the transducer case 52. An internal thread 62 to attach the proximal end of the probe 24 is formed at the distal end of the horn 60.

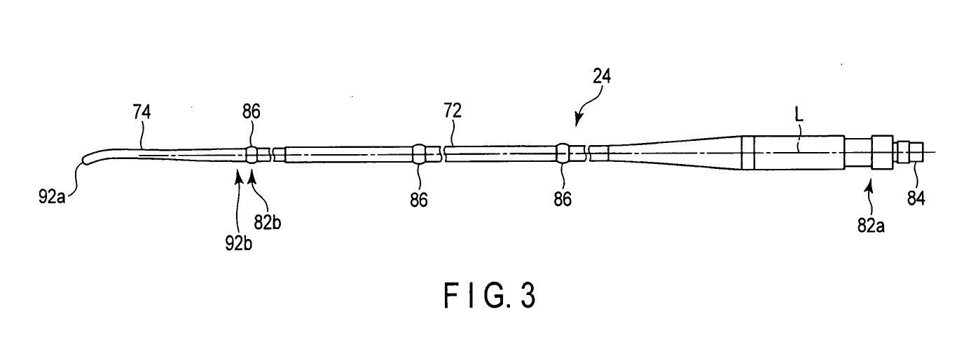

As shown in FIG. 3, the probe 24 according to this embodiment is made of, for example, titanium alloy and is rod-shaped. The probe 24 includes a probe body 72, and the treatment portion 74 which is provided on the distal side of the probe body 72 and which applies ultrasonic vibration to a living tissue while holding the living tissue together with the jaw 44 to treat the living tissue, such as by coagulation or cutting.

The probe body 72 includes a proximal end 82a and a distal end 82b. The proximal end 82a and the distal end 82b define a longitudinal axis L of the probe body 72. An external thread 84, which is screwed to the internal thread 62 of the ultrasonic transducer unit 28, is formed at the proximal end 82a of the probe body 72. If the external thread 84 is screwed to the internal thread 62 of the horn 60 in the ultrasonic transducer unit 28, the probe 24 and the ultrasonic transducer unit 28 are put together. Thus, if ultrasonic vibration is input to the proximal end 82a of the probe body 72 from the ultrasonic transducer 54 of the ultrasonic transducer unit 28, the ultrasonic vibration is transmitted from the proximal end 82a of the probe body 72 to the distal end 82b along the longitudinal axis L.

Supports 86 made of an electrically insulating ring-shaped elastic member are attached to all or some of node positions of vibration of the probe body 72 along its axial direction. The supports 86 are made of, for example, a rubber material. The supports 86 prevent interference between the outer circumferential surface of the probe body 72 and the inner circumferential surface of the sheath 42.

The treatment portion 74 is integrally formed on the distal side of the distal end 82b of the probe body 72. The treatment portion 74 is capable of holding the living tissue between the treatment portion 74 and the jaw 44. If ultrasonic vibration is input to the proximal end 82a of the probe body 72, the ultrasonic vibration is transmitted to the treatment portion 74 from the proximal end 82a of the probe body 72 via the distal end 82b. Thus, the treatment portion 74 is capable of treating the living tissue as described above, such as by coagulation or cutting.

As shown in FIG. 3 to FIG. 5C, the treatment portion 74 includes a distal end 92a opposite to the proximal end 82a of the probe body 72, a proximal end 92b opposite to the proximal end 82a of the probe body 72, a holding surface (surface located close to the jaw 44) 94 which holds the living tissue between the jaw 44 and the holding surface 94, a back surface 96 located on the rear surface (surface located away from the jaw 44) of the holding surface 94, and a side surface 98 which extend from the proximal end 92b to the distal end 92a and which is different from the holding surface 94.

The ultrasonic vibration generated from the ultrasonic transducer unit 28 is transmitted to the proximal end 92b. The ultrasonic vibration is transmitted to the distal end 92a through the proximal end 92b. The distal end 92a and the proximal end 92b are formed as continuous portions which are continuously formed between the holding surface 94 and the back surface 96. In this embodiment, the side surface 98 has first and second surfaces (side bodies) 98a and 98b which are each continuous between the holding surface (holding surface side region) 94 and the back surface (back surface side region) 96. That is, the side surface 98 preferably has a pair of side bodies 98a and 98b each having a curved surface between the holding surface 94 and the back surface 96.

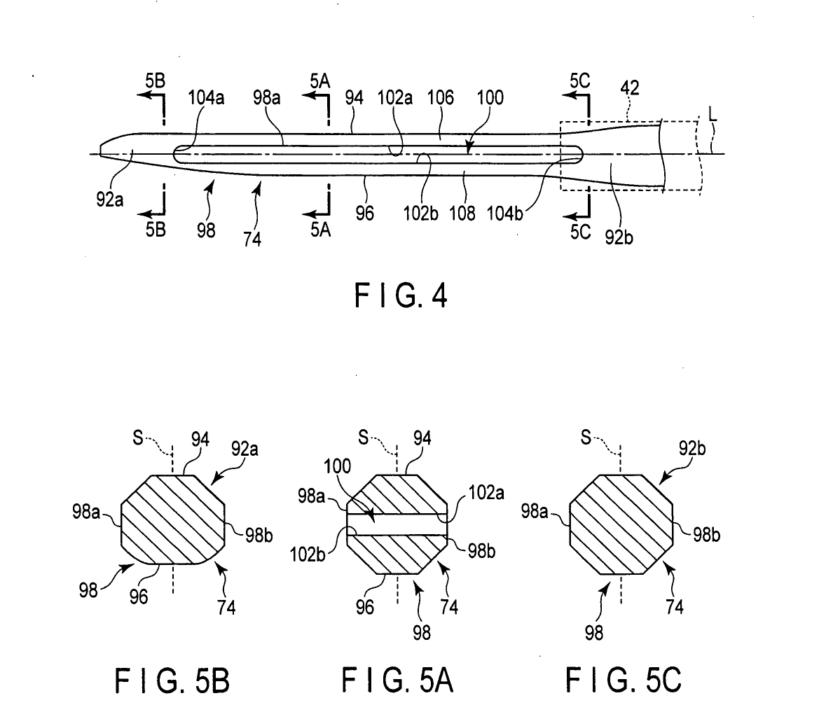

As shown in FIG. 4, a stress release portion (crack growth suppression portion) 100, which is slit-shaped in this embodiment, is formed. The stress release portion 100 releases concentrated stress between the distal end 92a and the proximal end 92b of the treatment portion 74 and on the side surface 98 to stop the development of a crack when the crack is produced in the holding surface 94 or the back surface 96 by external force applied to the treatment portion 74. In this embodiment, the stress release portion 100 is formed to pierce the pair of side bodies 98a and 98b that are located between the holding surface 94 which holds the living tissue between the jaw 44 and the holding surface 94, and the back surface 96 of the holding surface 94, as shown in FIG. 5A. Thus, the stress release portion 100 of the treatment portion 74 is formed as a discontinuous portion which is discontinuous along a rotational surface (pivot surface) S formed by the rotation of the jaw 44 (closer to or away from the treatment portion 74).

On the other hand, as shown in FIG. 5B, the distal end 92a of the treatment portion 74 is solidly formed without, for example, holes or slots in its cross section perpendicular to the longitudinal axis L. As shown in FIG. 5C, the proximal end 92b of the treatment portion 74 is solidly formed without, for example, holes or slots in its cross section perpendicular to the longitudinal axis L.

The stress release portion 100 according to this embodiment includes a pair of surfaces (crack growth control surfaces) 102a and 102b opposed to each other, a first semicircular portion 104a adjacent to the distal end 92a of the treatment portion 74, and a second semicircular portion 104b adjacent to the proximal end 92b of the treatment portion 74. The pair of opposed surfaces 102a and 102b of the stress release portion 100 intersect at right angles with a rotational surface S (see FIG. 5A to FIG. 5C) of the jaw 44 in this embodiment. The space between the pair of surfaces 102a and 102b is formed at a substantially equal distance along the longitudinal axis L. The first semicircular portion 104a (the distal end of the stress release portion 100) and the second semicircular portion 104b (the proximal end of the stress release portion 100) face each other. The stress release portion 100 according to this embodiment has a uniform width, and has curved surfaces formed at both ends (the distal end indicated by the sign 104a and the proximal end indicated by the sign 104b in FIG. 4).

In other words, the treatment portion 74 includes a holding surface side region 106 having the crack growth control surface 102a, and a back surface side region 108 having the crack growth control surface 102b. The holding surface side region 106 and the back surface side region 108 are coupled to each other by the distal end 92a and the proximal end 92b, but are discontinuous between the distal end 92a and the proximal end 92b.

As shown in FIG. 4, the proximal end 92b of the treatment portion 74 and the second semicircular portion 104b of the stress release portion 100 (the proximal end of the stress release portion 100) are located closer to the handle unit 22 than the supporting point 46 of the jaw 44 when the sheath unit 26 and the probe 24 are attached to the handle unit 22. Thus, the second semicircular portion 104b (the proximal end of the stress release portion 100) is located closer to the proximal side than the distal end of the sheath 42 along the longitudinal axis L.

The length of the half wavelength is about 51 mm to 52 mm when the resonant frequency (drive frequency) of the ultrasonic transducer 54 is 47 kHz, when the probe 24 is made of 6-4 Ti, and when the outside diameter of the probe body 72 is about 6 mm. Thus, when the external thread 84 of the probe 24 is joined to the internal thread 62 of the horn 60 at an antinode of vibration, the length from the distal end of the ultrasonic transducer 54 (the proximal end of the horn 60) to the distal end 92a of the treatment portion 74 is 51 mm to 52 mm×n (n: an integer equal to or more than 1). That is, the length from the distal end of the ultrasonic transducer 54 (the proximal end of the horn 60) to the distal end 92a of the probe 24 is the integral multiple (n-th) of the half wavelength.

The half wavelength is about 102 mm to 104 mm when the resonant frequency (drive frequency) of the ultrasonic transducer 54 is 23.5 kHz. In this case, the length from the distal end of the ultrasonic transducer 54 (the proximal end of the horn 60) to the distal end 92a of the treatment portion 74 is, for example, the integral multiple of 102 mm to 104 mm.

Next, the functions of the ultrasonic treatment instrument 12 according to this embodiment are described.

The ultrasonic transducer unit 28 and the sheath unit 26 are attached to the handle unit 22 at predetermined positions. The probe 24 is inserted through the sheath 42 of the sheath unit 26, and the external thread 84 at the proximal end 82a of the probe 24 is screwed and attached to the internal thread 62 of the horn 60 at the distal end of the ultrasonic transducer 54. The ultrasonic treatment instrument 12 according to this embodiment is used in this condition.

An input signal is input to the control section 14a of the power supply unit 14 by the input section 16 connected to the power supply unit 14, and one of the switches 38a and 38b is pressed in accordance with a treatment purpose. In response to this operation of one of the switches 38a and 38b, the control section 14a supplies electric power to the ultrasonic transducer 54 from the electric power supply 14b via the electric wiring lines 56a and 56b inside the cable 58. Accordingly, the ultrasonic transducer 54 ultrasonically vibrates. The ultrasonic vibration generated in the ultrasonic transducer 54 is increased in amplitude by the horn 60 and then transmitted to the probe body 72 and the treatment portion 74. When the ultrasonic vibration is transmitted to the probe 24 in this way, the probe 24 performs longitudinal vibration having a vibration direction and a transmission direction that are parallel to the longitudinal direction L.

In the handle unit 22, the movable handle 36 is forced away from the fixed handle 34 by a biasing force of an unshown spring. In this instance, the jaw 44 is forced away from the treatment portion 74 of the probe 24; in other words, the jaw 44 is open. If the movable handle 36 is moved closer to the fixed handle 34 against the biasing force of the unshown spring in this condition, the jaw 44 is moved closer to the treatment portion 74 of the probe 24 by a known mechanism; in other words, the jaw 44 is closed.

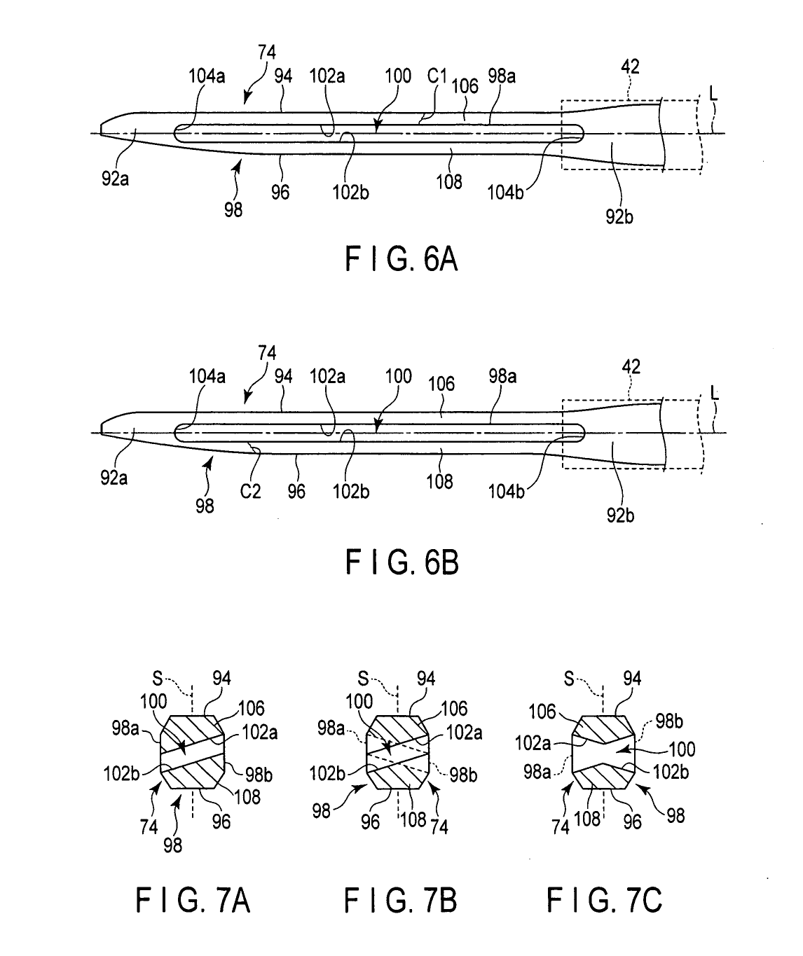

For example, when a living tissue is held between the jaw 44 and the holding surface 94 of the treatment portion 74 of the probe 24 so that the jaw 44 and the treatment portion 74 of the probe 24 are disposed in a body cavity, an unexpected overload may be applied to the holding surface 94 of the probe 24 by, for example, from griping a metallic material buried in the living tissue. When the overload is applied in this manner, a crack C1, such as a fracture or a flaw, may be produced in the holding surface side region 106 of the treatment portion 74 of the probe 24, for example, as shown in FIG. 6A.

The crack C1 is produced, for example, from the holding surface 94 toward the stress release portion 100. In this instance, the holding surface side region 106 and the back surface side region 108 are discontinuous because of the surfaces 102a and 102b. Thus, even if the crack C1 is produced in the treatment portion 74, the growth of the crack C1 is stopped by the crack growth control surface 102a of the stress release portion 100. That is, even if the crack C1 is produced in the treatment portion 74, the growth of the crack C1 is blocked by the stress release portion 100. Therefore, the growth of the crack C1 from the holding surface 94 to the back surface 96 can be prevented by the stress release portion 100. The treatment portion 74 is solidly formed on both the front side (the distal end 92a) and the rear side (the proximal end 92b) of the stress release portion 100 along the longitudinal axis L. Thus, even if the crack C1 is produced in the treatment portion 74, breaking and separation of the distal side of the treatment portion 74 are prevented. Therefore, separation of a part of the treatment portion 74 is prevented.

The crack C1 is not only stopped in the surface (crack growth control surface) 102a of the stress release portion 100 but is also stopped in the first semicircular portion 104a or the second semicircular portion 104b.

The treatment portion 74 projecting from the distal end of the sheath 42 in the probe 24 may contact other equipment (e.g. a trocar), and a load may be unexpectedly applied to treatment portion 74. When the load is applied in this manner, a crack C2, such as a fracture or a flaw, may be produced in the back surface side region 108 of the treatment portion 74 of the probe 24, for example, as shown in FIG. 6B.

The crack C2 is produced, for example, from the back surface 96 of the treatment portion 74 toward the stress release portion 100. In this instance, the holding surface side region 106 and the back surface side region 108 are discontinuous because of the surfaces 102a and 102b. Thus, even if the crack C2 is produced in the treatment portion 74, the growth of the crack C2 is stopped by the crack growth control surface 102b of the stress release portion 100. That is, even if the crack C2 is produced in the treatment portion 74, the growth of the crack C2 is blocked by the stress release portion 100. Therefore, the growth of the crack C2 from the back surface 96 to the holding surface 94 can be prevented by the stress release portion 100. The treatment portion 74 is solidly formed on both the front side (the distal end 92a) and the rear side (the proximal end 92b) of the stress release portion 100 along the longitudinal axis L. Thus, even if the crack C2 is produced in the treatment portion 74, breaking and separation of the distal side of the treatment portion 74 are prevented. Therefore, separation of a part of the treatment portion 74 is prevented.

The crack C2 is not only stopped in the surface (crack growth control surface) 102b of the stress release portion 100, but is also stopped in the first semicircular portion 104a or the second semicircular portion 104b.

Here, the proximal end of the stress release portion 100 is located closer to the proximal side than the distal end of the sheath 42 along the longitudinal axis L. Thus, even if the crack C1 or C2 is produced by the contact of other equipment with the distal end of the sheath 42, the growth of the crack C1 or C2 is blocked by the surface (crack growth control surface) 102a or 102b of the stress release portion 100. Thus, separation of the front part of the stress release portion 100 in the treatment portion 74 from the probe 24 can be prevented. Therefore, separation of a part of the treatment portion 74 is prevented.

If ultrasonic vibration is transmitted to the treatment portion 74 of the probe 24 in which the crack C1 or C2 is produced, the vibration significantly changes due to the crack C1 or C2 from a condition where the crack C1 or C2 is not present. Thus, the formation of the crack C1 or C2 in the treatment portion 74 is easily detected. Specifically, if ultrasonic vibration is generated from the ultrasonic transducer 54 in a condition where the crack C1 or C2 is produced in the treatment portion 74, it is expected that the frequency will become lower than the resonant frequency. Although such an event is unlikely in this embodiment, in event of the breaking of the treatment portion 74, it is expected that the frequency will become higher than the resonant frequency.

In this way, when the crack C1 or C2 is formed in the treatment portion 74, the change of the frequency of the ultrasonic vibration is reported to the control section 14a from the ultrasonic transducer 54 through the signal line inside the cable 58. In this instance, it is preferable that the control section 14a is programmed to control the electric power supply 14b and then immediately stop the supply of electric power to the ultrasonic transducer 54. In other words, the ultrasonic transducer 54 detects an abnormality in the probe 24 by the vibration, and if a signal is input to the control section 14a from the ultrasonic transducer 54, the control section 14a automatically stops the supply of electric power to the ultrasonic transducer 54. When the treatment portion 74 is located in the body cavity, the treatment portion 74 is slowly taken out of the body cavity.

As described above, the following can be said according to this embodiment.

The probe 24 according to this embodiment when in use is attached to the treatment instrument body 20, which has the jaw 44 rotatable by the supporting point 46 and the handle unit 22 provided on the proximal side of the jaw 44. The probe 24 includes the probe body 72 and the treatment portion 74. The probe body 72 includes the proximal end 82a, the distal end 82b, and the longitudinal axis L defined by the proximal end 82a and the distal end 82b. If ultrasonic vibration is input to the proximal end 82a from the ultrasonic transducer unit 28 attached to the treatment device body 20, the ultrasonic vibration is transmitted from the proximal end 82a toward the distal end 82b along the longitudinal axis L. The treatment portion 74 is capable of holding a living tissue between the treatment portion 74 and the jaw 44, and treating the living tissue to which ultrasonic vibration is transmitted from the proximal end 82a via the distal end 82b along the longitudinal axis L when the ultrasonic vibration is input to the proximal end 82a of the probe body 72. The treatment portion 74 includes the distal end 92a which is provided on the distal side of the distal end 82b of the probe body 72 and which is opposite to the proximal end, the proximal end 92b opposite to the proximal end 82a, the holding surface 94 which holds the living tissue between the holding surface 94 and the jaw 44, the back surface 96 of the grasp surface 94, and the pair of side bodies 98a and 98b located between the holding surface 94 and the back surface 96. The treatment portion 74 is located between the distal end 92a and the proximal end 92b and between the side bodies 98a and 98b, and when external force is applied to the treatment portion 74 and a crack is produced in the holding surface 94 or the back surface 96, the treatment portion 74 can guide the tip (stress concentrated portion) of the crack to the stress release portion 100. In other words, the stress release portion 100 concentrates stress at the tip of the crack produced in the holding surface 94 to release the stress at the tip of the crack which has developed from the holding surface 94 to the back surface 96, and can then stop the development of the crack. Alternatively, the stress release portion 100 concentrates stress at the tip of the crack produced in the back surface 96 to release the stress at the tip of the crack which has developed from the back surface 96 to the holding surface 94, and can then stop the development of the crack. Specifically, the stress release portion 100 including the pair of crack growth control surfaces 102a and 102b is formed in the treatment portion 74 of the probe 24 in a direction tilted relative to the open-close direction (rotation surface S) of the jaw 44; here in particular, in a direction which intersects at right angles. Thus, even if the crack C1 is produced from the holding surface 94 of the treatment portion 74, the crack C1 grows toward the stress release portion 100, so that the growth of the crack C1 up to the back surface 96 can be prevented by the stress release portion 100. Even if the crack C2 is produced from the back surface 96 of the treatment portion 74, the crack C2 grows toward the stress release portion 100, so that the growth of the crack C2 up to the holding surface 94 can be prevented by the stress release portion 100. Therefore, the treatment portion 74 according to this embodiment allows the stress release portion 100 to stop the growth, that is, the development of a crack from the holding surface 94 to the back surface 96 or from the back surface 96 to the holding surface 94.

When the probe 24 is attached to the treatment instrument body 20, the proximal end 92b of the treatment portion 74 is located closer to the treatment instrument body 20 than the distal end of the sheath 42 or the supporting point 46 of the jaw 44. When the probe 24 is attached to the treatment instrument body 20, the proximal end, that is to say, a part of the stress release portion 100, is located closer to the handle unit 22 than the distal end of the sheath 42 or the supporting point 46 of the jaw 44. Thus, the position closer to the proximal side than the distal end of the sheath 42 along the longitudinal axis L is protected by the sheath 42. Therefore, the occurrence of a crack from the holding surface 94 or the back surface 96 at the proximal end 92b of the treatment portion 74 can be prevented.

In this embodiment, as shown in FIG. 5A, the pair of opposed crack growth control surfaces 102a and 102b of the stress release portion 100 are described as the surfaces that intersect at right angles with the rotation surface S of the jaw 44. However, as shown in FIG. 7A to FIG. 7C, the crack growth control surfaces 102a and 102b may be formed as inclined surfaces that are inclined relative to the surfaces that intersect at right angles with the rotation surface S of the jaw 44. It is also preferable that the pair of surfaces (crack growth control surfaces) 102a and 102b are formed as curved surfaces instead of flat surfaces.

In the embodiment described above, the pair of opposed crack growth control surfaces 102a and 102b intersect at right angles with the rotation direction of the jaw 44. However, as shown in FIG. 7A to FIG. 7C, it is also preferable that the pair of crack growth control surfaces 102a and 102b are formed as surfaces which are inclined relative to the rotation direction of the jaw 44 and relative to the direction that intersects at right angles with the rotation direction.

FIG. 7A shows how the side bodies 98a and 98b of the treatment portion 74 are pierced by, for example, laser processing or electric-discharge machining. A broken line in FIG. 7B indicates a part to be removed by, for example, laser processing or electric-discharge machining. FIG. 7C shows an example of the stress release portion 100 that is formed by, for example, laser processing or electric-discharge machining.

The probe 24 which includes the treatment portion 74 having the stress release portion 100 shown in FIG. 7A may also be used.

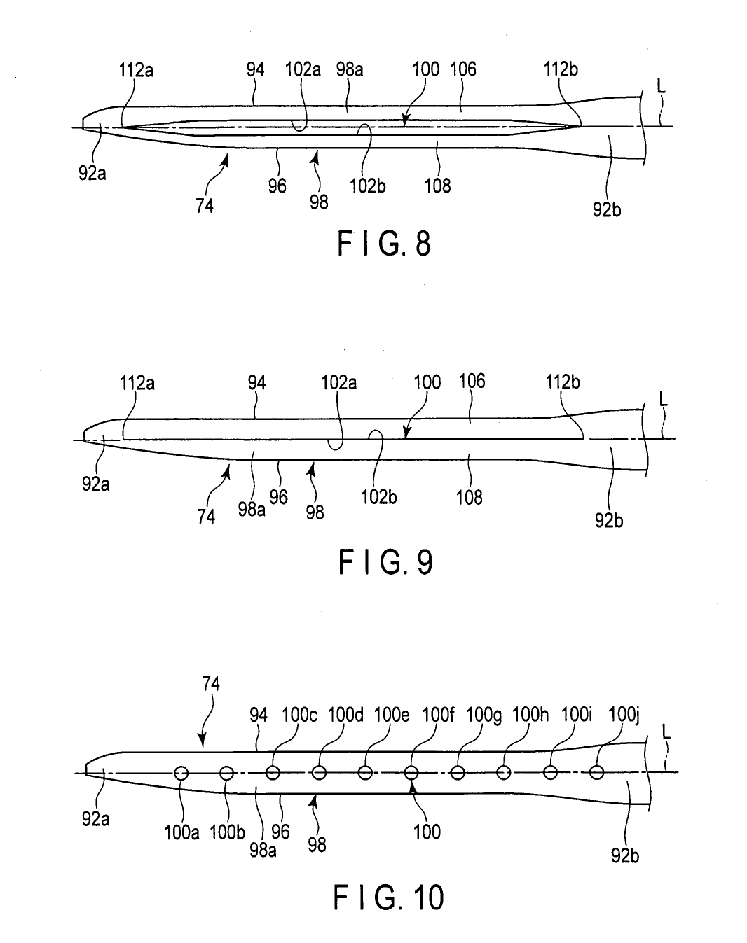

The pair of opposed crack growth control surfaces 102a and 102b shown in FIG. 7A may, of course, be in abutment with each other as appropriate. Next, a first modification of the first embodiment is described with reference to FIG. 8. In the slit stress release portion 100 according to this embodiment, the shape of the first semicircular portion 104a close to the distal end 92a of the treatment portion 74 and the shape of the second semicircular portion 104b close to the proximal end 92b are modified.

As shown in FIG. 8, the first semicircular portion 104a adjacent to the distal end 92a of the treatment portion 74 of the probe 24 is modified to an acute portion 112a. Similarly, the second semicircular portion 104b adjacent to the proximal end 92b of the treatment portion 74 of the probe 24 is modified to an acute portion 112b. Thus, the stress release portion 100 according to this modification is formed so that the width between the pair of surfaces 102a and 102b gradually decreases from the pair of opposed crack growth control surfaces 102a and 102b toward the acute portions 112a and 112b. Therefore, in the stress release portion 100 of the treatment portion 74 according to this modification, the sectional change of the cross section in the longitudinal direction L is more gradual than in the stress release portion 100 according to the first embodiment shown in FIG. 4. When the stress release portion 100 is formed in this way, stress concentration caused by vibration at both ends 112a and 112b of the stress release portion 100 in the longitudinal direction can be lessened, and the structural strength of the treatment portion 74 can be maintained at a higher level than that of the treatment portion 74 described in the first embodiment.

The crack C1 or C2 is caused toward the stress release portion 100 as shown in FIG. 6A and FIG. 6B in the first embodiment.

Next, a second modification of the first embodiment is described with reference to FIG. 9. In this embodiment, the shape of the stress release portion 100 according to the first modification is further modified.

As shown in FIG. 9, there is no space (width) between the pair of opposed crack growth control surfaces 102a and 102b of the stress release portion 100, in contrast with the first embodiment shown in FIG. 4 and the first modification of the first embodiment shown in FIG. 8. Thus, the structural strength of the treatment portion 74 can be maintained at a higher level than that of the treatment portion 74 described in the first embodiment and that of the treatment portion 74 described in the first modification. The functions and advantageous effects described in the first embodiment are also provided even in this condition.

The stress release portion 100 according to this modification is suitably formed. For example, the slit stress release portion 100 according to the first modification is formed and is then pressed so that the pair of opposed crack growth control surfaces 102a and 102b of the stress release portion 100 are brought into contact with each other, or different components are put together by various means.

Next, a third modification of the first embodiment is described with reference to FIG. 10. In this embodiment, the shape of the stress release portion 100 is modified. Although one stress release portion is provided in the embodiment including the modifications described above by way of example, more than one stress release portion are provided in this modification that will be described by way of example.

As shown in FIG. 10, the treatment portion 74 includes openings 100a through 100j as the stress release portions. These openings 100a through 100j are arranged between the distal end 92a and the proximal end 92b of the treatment portion 74 along the longitudinal axis L. It is preferable that the openings 100a through 100j are, for example, equally spaced. Each of the openings 100a through 100j pierces the side bodies 98a and 98b of the treatment portion 74. The edge of each of the openings 100a through 100j is circular. The central axis of each of the openings 100a through 100j extends in a direction that intersects at right angles with the longitudinal axis L, and intersects at right angles with the surface formed by the rotation surface S of the jaw 44.

When external force is applied to the holding surface 94 of the treatment portion 74 and causes a crack in the holding surface 94, the crack extends to one of the openings 100a through 100j from the holding surface 94. When external force is applied to the back surface 96 of the treatment portion 74 and causes the back surface 96 to crack, the crack extends to one of the openings 100a through 100j from the back surface 96. In other words, when external force is applied to the treatment portion 74 and causes the treatment portion 74 to crack, depending on the conditions at the time, the stress is released in one of the openings 100a through 100j. Therefore, the region of the treatment portion 74 where the openings 100a through 100j are distributed is more easily broken than the solid distal end 92a and proximal end 92b of the treatment portion 74. Thus, even if the back surface 96 has cracked from the holding surface 94 of the treatment portion 74 or the holding surface 94 has cracked from the back surface 96, the crack does not pass between the openings, for example, between the openings 100c and 100d. Instead, the crack is guided and extends to one of the openings 100a through 100j from the holding surface 94 or the back surface 96, and is then blocked at the edge of one of the openings 100a through 100j.

The shape of the edge of each of the openings 100a through 100j is not exclusively circular, and may be, for example, substantially elliptical. The ellipse can be formed, for example, by laser processing or by pressing the treatment portion 74 after forming a circular edge.

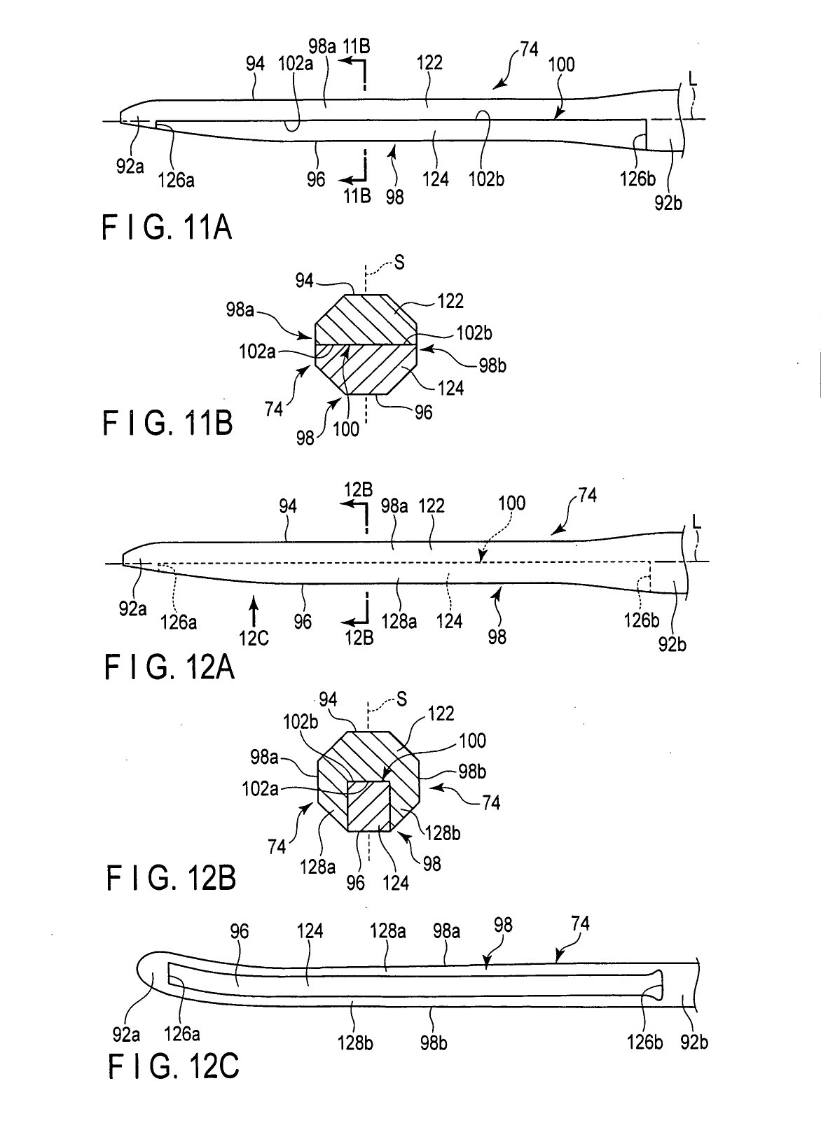

Next, a second embodiment is described with reference to FIG. 11A and FIG. 11B. This embodiment is a modification of the first embodiment including the first to third modifications. The parts having the same functions as those according to the first embodiment are denoted by the same reference signs, thus a detailed description thereof is omitted.

As shown in FIG. 11A, the holding surface 94 and the back surface 96 are formed as independent components in the treatment portion 74 of the probe 24 according to this embodiment. In other words, in the treatment portion 74, a holding surface side region 122 and a back surface side region 124 that are separate from each other are integrally formed at the distal end 92a and the proximal end 92b of the treatment portion 74. Here, the side surface 98 includes the back surface side region 124.

In this embodiment, the holding surface side region 122 including the holding surface 94 of the treatment portion 74 is made of, for example, a titanium alloy. The back surface side region 124 including the back surface 96, particularly the side surface 98 of the treatment portion 74, is made of an aluminum alloy or a resin material such as engineering plastic. For example, a heat-resistant material such as a fluorocarbon resin material or a PEEK material is used as the engineering plastic. It is also preferable that the back surface side region 124 has electrically insulating properties.

When both the holding surface side region 122 and the back surface side region 124 are made of a metallic material, welding, press-shaping, cladding, insert molding, or press fitting, for example, can be used. When the grasp surface side region 122 is made of a metallic material and the back surface side region 124 is made of a resin material such as the engineering plastic, the insert molding or the press fitting can be used.

In this embodiment, as shown in FIG. 11A and FIG. 11B, the distal end of the back surface side region 124 is attached to an attachment portion 126a at the distal end 92a of the treatment portion 74, and the proximal end of the back surface side region 124 is attached to an attachment surface 126b at the proximal end 92b of the treatment portion 74. Here, the distal end of the back surface side region 124 and the attachment portion 126a at the distal end 92a of the treatment portion 74 are shaped to fit into each other. The proximal end of the back surface side region 124 and the attachment surface 126b at the proximal end 92b of the treatment portion 74 are shaped not only to fit into each other but also to engage with each other. The pair of opposed surfaces (crack growth control surfaces) 102a and 102b are provided at the boundary between the holding surface side region 122 and the back surface side region 124.

When external force is applied to the holding surface 94 of the treatment portion 74 and causes the holding surface 94 to crack, further development of the crack is stopped by the crack growth control surface 102a of the stress release portion (crack growth control portion) 100. However, the distal end 92a of the holding surface side region 122 is separated from the proximal end 92b. Here, the distal end 92a of the treatment portion 74, in other words the distal end 92a of the holding surface side region 122, is attached to the distal end of the back surface side region 124, and the proximal end 92b of the treatment portion 74, in other words, the proximal end 92b of the holding surface side region 122, is attached to the proximal end of the back surface side region 124. Thus, separation of the distal end 92a of the holding surface side region 122 from the treatment portion 74 is prevented.

When external force is applied to the back surface 96 of the treatment portion 74 and causes the back surface 96 to crack, further development of the crack is stopped by the crack growth control surface 102b of the stress release portion 100. However, the distal end of the back surface side region 124 is separated from the proximal end thereof. Here, the distal end of the back surface side region 124 is attached to the attachment surface 126a, and the proximal end is attached to the attachment surface 126b. Thus, separation of the distal end of the back surface side region 124 from the treatment portion 74 is prevented.

Even if different components are used in the holding surface side region 122 and the back surface side region 124 as in this embodiment, it is possible to stop the development of the crack and prevent the separation of the component from the treatment portion 74 as in the first embodiment including the modifications.

Although not shown, the pair of opposed crack growth control surfaces 102a and 102b may be spaced out as has been described in the first embodiment.

Next, a first modification of the second embodiment is described with reference to FIG. 12A to FIG. 12C.

As shown in FIG. 12A to FIG. 12C, the cross section of the holding surface side region 122 of the treatment portion 74 according to this modification is substantially U-shaped. Thus, the stress release portion 100 is provided on the side surface 98, but does not pierce the side bodies 98a and 98b. That is, the side surface 98 has a pair of side surfaces 128a and 128b in the holding surface side region 122. Thus, the flexural rigidity of the holding surface side region 122 can be increased by the side surfaces 128a and 128b.

As shown in FIG. 12C, it is also preferable that the distal end of the back surface side region 124 is bent and that the proximal end of the back surface side region 124 is formed with a lager size than the width between the distal end and the proximal end. Thus, the back surface side region 124 is not only attached to, but also engaged with the holding surface side region 122. This further ensures that the separation of the component from the treatment portion 74 can be prevented in comparison to when the back surface side region 124 is only fixed to the holding surface side region 122. In this embodiment, as shown in FIG. 12C, the proximal end of the back surface side region 124 and the attachment portion 126b at the proximal end 92b of the treatment portion 74 are structured to be not only fitted to, but also engaged with each other. It is also preferable that the distal end of the back surface side region 124 and the attachment surface 126a at the distal end 92a of the treatment portion 74 are structured to be not only fitted to, but also engaged with each other.

The movable handle 36 is disposed in front of the fixed handle 34 in the treatment instrument 12 according to the first embodiment including the modifications and the second embodiment including the first modification described above by way of example. The movable handle 36 may be disposed in the rear of the fixed handle 34. It is also preferable that the fixed handle 34 and the movable handle 36 are disposed at opposite positions relative to the longitudinal axis L. That is, the treatment instrument body 20 is not limited to the shape shown in FIG. 1.

Ultrasonic vibration is transmitted to the probe 24 in the first and second embodiments including the modifications described above by way of example. The ultrasonic treatment system 10 may be configured so that ultrasonic vibration is transmitted to the probe 24 and/or so that high-frequency energy is applied to a living tissue between the jaw 44 and the treatment portion 74 of the probe 24 instead of the ultrasonic vibration.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A probe configured to treat a living tissue in cooperation with a jaw which is configured to pivot on a supporting point, the probe comprising a treatment portion,
the treatment portion comprising:
a proximal end to which ultrasonic vibration generated from an ultrasonic transducer unit is transmitted;
a distal end to which the ultrasonic vibration is transmitted from the proximal end;
a holding surface configured to hold the living tissue between the holding surface and the jaw;
a back surface serving as a rear surface of the holding surface;
a side surface which extends from the proximal end to the distal end and which is different from the holding surface; and
a stress release portion which is provided between the distal end and the proximal end and on the side surface and which releases stress to stop development of a crack when the crack is produced in the holding surface or the back surface by external forces applied during the treatment of the living tissue.

2. The probe according to claim 1, wherein
the distal end and the proximal end are formed as continuous portions which are continuously formed between the holding surface and the back surface, and
the stress release portion is formed as a discontinuous portion which is discontinuously formed between the holding surface and the back surface.

3. The probe according to claim 2, wherein
the side surface includes first and second surfaces which are each continuous between the holding surface and the back surface, and
the discontinuous portion is formed to pierce the first and second surfaces.

4. The probe according to claim 3, wherein the discontinuous portion includes a pair of inclined surfaces which are inclined relative to a pivot surface defined by the pivoting of the jaw on the supporting point.

5. The probe according to claim 4, wherein the pair of inclined surfaces are located between the holding surface and the back surface and extend along an axial direction of a longitudinal axis defined by the distal end and the proximal end.

6. The probe according to claim 3, wherein the discontinuous portion includes a pair of surfaces which face each other and which are separate from each other.

7. The probe according to claim 3, wherein the discontinuous portion includes a pair of surfaces which face each other and which are in abutment with each other.

8. The probe according to claim 2, wherein
the holding surface and the back surface are made of different materials, and
the discontinuous portion serves as a boundary between the different materials.

9. The probe according to claim 1, wherein
the side surface includes first and second surfaces which are each continuous between the holding, surface and the back surface, and
the stress release portion includes openings respectively piercing the first and second surfaces.

10. A treatment instrument comprising:
a treatment instrument body including a jaw which is configured to pivot on a supporting point; and
the probe according to claim 1 which is attachable to the treatment instrument body and which is configured to treat a living tissue together with the jaw by a pivot of the jaw.

11. The treatment instrument according to claim 10, wherein
the treatment instrument body includes a handle unit provided on the proximal side of the jaw,
the proximal end of the probe is located closer to the treatment instrument body than the supporting point of the jaw when the probe is attached to the treatment instrument body, and
at least a part of the stress release portion is located closer to the handle unit than the supporting point of the jaw when the probe is attached to the treatment instrument body.

12. The treatment instrument according to claim 10, wherein
the treatment instrument body includes a sheath which covers the outer circumferential surface of the proximal end of the treatment portion of the probe and on which the jaw and the supporting point are supported, and a handle unit provided on the proximal side of the sheath,
the proximal end of the treatment portion is located closer to the handle unit than the distal end of the sheath when the probe is attached to the treatment instrument body, and
at least a part of the stress release portion is located closer to the handle unit than the distal end of the sheath when the probe is attached to the treatment instrument body.

13. The treatment instrument according to claim 10, further comprising an ultrasonic transducer unit configured to input ultrasonic vibration to the probe attached to the treatment instrument body.
